(19) United States
(12) Patent Application Publication
(10) Pub. No.: US 2026/0053881 A1
(43) Pub. Date: Feb. 26, 2026

(54) Supplement Formulation for Enhanced Bioavailability of Turmeric Extract Focusing on Ease of Use

(71) Applicant: Caitlin Ferzli, New York, NY (US)

(72) Inventor: Caitlin Ferzli, New York, NY (US)

(21) Appl. No.: 18/813,025

(22) Filed: Aug. 23, 2024

(51) Int. Cl. A61K 36/9066; A61K 31/12; A61K 47/02; A61K 47/12; A61K 47/36

(52) U.S. Cl. CPC A61K 36/9066; A61K 31/12; A61K 47/02; A61K 47/12; A61K 47/36

(57) ABSTRACT

Aspects of the present invention relate to a dietary supplement formulation designed to enhance the bioavailability of turmeric extract using a specific combination of soluble fibers and natural sweeteners. The formulation supports overall health by providing an effective dose of turmeric extract in a convenient stick pack format.

### BACKGROUND

Turmeric extract, particularly its active component curcumin, is well-known for its anti-inflammatory and antioxidant properties. However, curcumin has low bioavailability due to poor solubility and rapid metabolism. Various formulations have been developed to address this issue, but there remains a need for a convenient, effective, and consumer-friendly product.

### SUMMARY OF INVENTION

Thus, the present invention provides a formulation which enhances curcumin absorption while providing additional health benefits from dietary fiber.

### DETAILED DESCRIPTION

### Ingredients

Turmeric extract complex (rhizome): 300 mg
Source: Curcuma longa, standardized for enhanced bioavailability.
Soluble Corn Fiber: Provides dietary fiber and supports digestive health.
Citric Acid: Used as a flavor enhancer and preservative.
Erythritol: A natural sweetener with low glycemic impact.
Natural flavors: Added to improve the taste of the supplement.
Silica: Used as an anti-caking agent.
Rebaudioside A: A steviol glycoside used as a sweetener.

### Formulation Process

### Mixing:

Combine turmeric extract complex with soluble corn fiber in a dry blending process to ensure even distribution.
Add citric acid, erythritol, natural flavors, silica, and rebaudioside A to the mixture.
Mix thoroughly to create a homogenous powder blend.

### Packaging:

The blended powder is portioned into individual stick packs, each containing 5 grams of the formulation.
Stick packs are sealed to ensure freshness and prevent moisture ingress.

### Quality Control:

Conduct tests to verify the uniformity of the active ingredient (turmeric extract) in each stick pack.
Test for stability, ensuring that the formulation maintains its efficacy and taste over the product's shelf life.

### Experimental Data

Preliminary testing indicates that the formulation significantly improves the bioavailability of turmeric extract compared to standard turmeric supplements. The inclusion of soluble corn fiber and specific natural sweeteners enhances the overall consumer experience and compliance.

Thus, the present invention provides a novel supplement formulation that enhances the bioavailability of turmeric extract while providing additional health benefits from dietary fiber. The described formulation process and preliminary data support the efficacy of this innovative product.

Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A dietary supplement formulation comprising:
Turmeric extract complex (rhizome);
Soluble corn fiber;
Citric acid;
Erythritol;
Natural flavors;
Silica; and
Rebaudioside A.

2. The formulation of claim 1 wherein the turmeric extract is standardized for enhanced bioavailability.

3. The formulation of claim 1 wherein the dietary fiber supports digestive health.

4. The formulation of claim 1 provided in a convenient stick pack format for ease of use.
